(19) United States
(12) Patent Application Publication
(10) Pub. No.: US 2021/0370244 A1
(43) Pub. Date: Dec. 2, 2021

(54) SYSTEMS AND METHODS FOR CONTROLLED DEVELOPMENT AND DELIVERY OF GAS AND LIQUID MIXTURES

(71) Applicant: BlueInGreen LLC, Fayetteville, AR (US)

(72) Inventor: Christopher Blake Milligan, Fayetteville, AR (US)

(21) Appl. No.: 17/331,681

(22) Filed: May 27, 2021

Related U.S. Application Data
(60) Provisional application No. 63/031,940, filed on May 29, 2020.

(51) Int. Cl. B01F 3/04; B01F 5/04; B01F 5/06; A61L 2/26; A61L 2/18; C02F 1/72; C02F 1/78; C02F 3/12

(52) U.S. Cl. CPC B01F 3/0446; B01F 3/04978; B01F 5/0403; B01F 5/0603; A61L 2101/02; A61L 2/18; C02F 1/727; C02F 1/78; C02F 3/1284; A61L 2/26

(57) ABSTRACT

Disclosed is a system for mixing gases and liquids that includes a reactor vessel and an injection assembly. The reactor vessel including a liquid inlet which receives a predetermined amount of liquid and at least one gas inlet which receives a precise amount of a gas. The reactor vessel also includes means for creating cavitation or turbulence for mixing the gas and liquid to a desired gas concentration.

### CLAIM OF PRIORITY

This application claims priority to U.S. Provisional Patent Application No. 63/031,940, filed May 29, 2020, the contents of which are hereby incorporated by reference.

### BACKGROUND OF THE INVENTION

### 1. Field of the Invention

The subject disclosure relates to systems and methods for the controlled development and delivery of gas and liquid mixtures, including sub saturated, saturated, and supersaturated solutions.

### 2. Background of the Related Art

Many different systems and methods, depending on application, are available for dissolving gases in liquids. Some of the main applications for using such systems are in the areas of water and wastewater treatment for municipal, commercial, and industrial sites; aquaculture; ground water remediation; ecological restoration and preservation; beverage making and bottling, and agriculture. Most of these traditional dissolved gas delivery systems (i.e., bubble diffusion, Venturi injection, U-tubes, Speece cones) attempt to leverage Henry's Law to achieve a high concentration of dissolved gas in the carrier liquid. These systems/methods typically require high flow rates and/or high operating pressures in order to achieve the desired amount of gas dissolution.

Many of these technologies provide energy input into the liquid and/or gas (e.g., via pumping) to achieve the high flow rates or operating pressure. For example, U.S. Pat. No. 9,315,402 discloses is a system and method for treating wastewater which includes a pressure vessel in which a gas is dissolved into the wastewater. The wastewater is supplied to the pressure vessel through a spray nozzle using a pumping mechanism.

Like most of the prior art systems, the system disclosed in U.S. Pat. No. 9,315,402, relies on the intense pressures within the pressure vessel to achieve high gas concentrations. While higher operating pressures lead to higher gas concentrations, achieving these higher pressures using a pumping mechanism is costly and may not be possible in certain applications where power is limited, for example.

Therefore, there is a need for simplified, low cost, systems and methods for dissolving a gas into a liquid. More specifically, there is a need for systems and methods which provide for more efficient and cost-effective water and wastewater treatment, enhance production capabilities for indoor and outdoor agriculture and aquaculture, and offer highly-effective disinfection capabilities for numerous municipal and industrial activities.

### SUMMARY OF THE INVENTION

The present disclosure is directed to systems and methods for the controlled development and delivery of gas and liquid mixtures, including sub saturated, saturated, and supersaturated solutions. The first component of which, the development phase, involves the mixing and pressurization of the gas and liquid at specific rates and volume ratios in order to achieve a target saturation level with a known concentration of dissolved and undissolved gas. The second component, the delivery phase, encompasses key design parameters for the delivery of gas and liquid mixtures to meet specific targeted concentrations and objectives, including enhancing hydrodynamic cavitation and increasing hydroxyl-radical formation.

It should be appreciated that the present invention can be implemented and utilized in numerous ways, including without limitation as a process, an apparatus, a system, a device, and a method for applications now known and later developed. These and other unique features of the systems and methods disclosed herein will become more readily apparent from the following description and the accompanying drawings.

### BRIEF DESCRIPTION OF THE DRAWINGS

So that those having ordinary skill in the art to which the disclosed systems and methods appertain will more readily understand how to make and use the same, reference may be had to the drawings wherein:

FIG. 1 provides a representation of the operational steps/phases used in a method for the controlled development and delivery of gas and liquid mixtures performed in accordance with an embodiment of the present invention;

FIG. 2A illustrates a serpentine reactor design which can be used during step 1 of the process of FIG. 1;

FIG. 2B illustrates a downflow reactor design which can be used during step 1 of the process of FIG. 1;

FIG. 2C illustrates a inline reactor design which can be used during step 1 of the process of FIG. 1;

FIG. 3 provides a representation of step 2 of the method of FIG. 1;

FIG. 4A illustrates an embodiment of a device which can be used to create cavitation in the flow during step 2 of the FIG. 1 process;

FIG. 4B illustrates a second embodiment of a device which can be used to create cavitation in the flow during step 2 of the FIG. 1 process;

FIG. 4C illustrates an entrainment collar which can be used for mixing the solution with a bulk fluid or process during step 2 of the FIG. 1 process; and

FIG. 5 illustrates a mixing nozzle which can be used to create cavitation or turbulence in the flow.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment

### DETAILED DESCRIPTION OF PREFERRED EMBODIMENTS

Disclosed herein are detailed descriptions of specific embodiments of the systems and methods for the controlled development and delivery of gas and liquid mixtures. It will be understood that the disclosed embodiments are merely examples of the way in which certain aspects of the invention can be implemented and do not represent an exhaustive list of all of the ways the invention may be embodied. Indeed, it will be understood that the systems, devices and methods described herein may be embodied in various and alternative forms some of which are described herein. Moreover, as noted above, the figures are not necessarily to scale and some features may be exaggerated or minimized to show details of particular components.

Well-known components, materials or methods are not necessarily described in great detail in order to avoid obscuring the present disclosure. Any specific structural and functional details disclosed herein are not to be interpreted as limiting, but merely as a basis for the claims and as a representative basis for teaching one skilled in the art to variously employ the invention.

Unless otherwise apparent, or stated, directional references, such as “right,” “left,” “upper,” “lower,” “outward,” “inward,” etc., are intended to be relative to the orientation of a particular embodiment of the invention as shown in the first numbered view of that embodiment. In addition, a given reference numeral indicates the same or similar structure when it appears in different figures and like reference numerals identify similar structural elements and/or features of the subject invention.

The present disclosure now will be described more fully, but not all embodiments of the disclosure are necessarily shown. In addition, many modifications may be made to adapt a particular situation or material to the teachings of the disclosure without departing from the essential scope thereof.

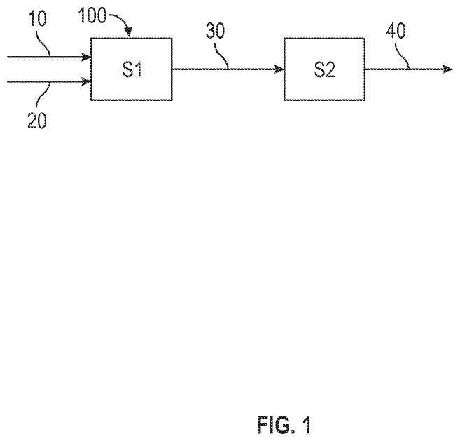

Referring now to FIGS. 1-5 which disclose several embodiments of the systems and methods for the controlled development and delivery of gas and liquid mixtures of the present invention. In each embodiment, the systems and methods are adapted to create a solution of gas and liquid in a controlled process and deliver that solution to an application point for benefit.

FIG. 1 provides a representation of the operational steps used in a method for the controlled development and delivery of gas and liquid mixtures performed in accordance with an embodiment of the present invention. As shown in this figure, liquid 10 and gas 20 are supplied to a pressure vessel 100. In Step 1 (S1) the liquid 10 is moved through the reactor/vessel 100 that is pressurized and gas 20 is mixed into the liquid via turbulence or cavitation within the vessel 100. The process is controlled such that the gas and liquid solution 30 leaving the pressure vessel 100 has a known concentration, generally described as subsaturated, saturated, or supersaturated.

The desired concentration of the solution is selected, calculated and known based on the particular application and process. An exact and precise amount of water/liquid is input into a pressurized vessel via gravity or pumping. An exact and precise amount of a particular gas is input into the same pressurized vessel via vacuum or positive pressure. The resulting solution has the exact and precise concentration desired. The solution is then maintained at the desired concentration until delivered back to the process in Step 2 (S2) via an injection assembly through outlet 40. The resulting solution is controlled to maximize at least one of the following: resulting bubble size (no bubbles, Nano bubbles, fine bubbles, etc.); resulting hydroxyl radical formation; and targeted treatment objective. It will readily be appreciated that the system could be operated in a continuous or batch mode.

Preferably, the vessel 100 must have at least 1 atmosphere of pressure. The water and gas must be mixed within the vessel such that a sufficient gas-to-liquid surface is achieved. The pressure inside the vessel must be maintained until the solution is delivered back to the process in Step 2 via the injection assembly.

The system leverages fundamental physical and chemical principles to create subsaturated, saturated, and supersaturated solutions. The governing principle is Henry's law which can be summarized as the concentration of a gas in a liquid is directly proportional to the pressure of the gas and liquid at steady state. However, the system does not operate under steady state assumptions. In the case of the presently disclosed system, the gas-to-liquid ratio, induced turbulence (discussed hereinwbelow), and pressure of the reaction is controlled such that solutions of varying concentrations can be generated. Similar to a natural stream with turbulence, it is possible to create a supersaturated solution, i.e. greater than 100% of steady state saturation according to Henry's law using cavitation. Similarly, the present system is a dynamic process, not steady state, and therefore the system allows for development of super-saturated solutions.

Unlike the present system, prior art systems are generally optimized to provide only saturated solutions. In the case of U.S. Pat. No. 9,315,402, the pressure vessel reactions are optimized to achieve a 100% saturated solution through the use of a spray nozzle which has significant cost implications due to induced pressure loss across the nozzle. Similarly, in other prior art constructions, the pressure vessel reactions are optimized to achieve a 100% saturated solution through the use of a ‘cone’ shaped vessel which creates different velocity profiles in order to suspend gas bubbles until they dissolve. Moreover, none of the prior art solutions describe the hydrodynamic cavitation principles or enhancement of hydroxyl radical formation at the injection assembly of the present system.

The capability to induce controlled hydrodynamic cavitation, in the case of the present systems and methods, is critical for delivery of supersaturated solutions while simultaneously achieving very high transfer efficiencies into various processes. The hydrodynamic cavitation allows for any gas that might remain in gaseous form from the vessel to the injection assembly, or downstream of the injection assembly, to be subjected to high energy gradients and shearing forces such that it instantly dissolves into the bulk fluid at the point of injection. Furthermore, the present systems and methods can be controlled to enhance formation of hydroxyl radicals at the injection assembly. By subjecting liquid to controlled hydrodynamic cavitation, formation of hydroxyl radicals is possible. Hydroxyl radicals have the highest oxidation potential currently understood which makes them extremely effective at disinfection. Operation of the present systems and methods can be such that the formation of these hydroxyl radicals is controlled. Furthermore, the formation of hydroxyl radicals can be enhanced by using oxygen or ozone as the source gas. Similarly, by utilizing oxygen (O2) as a source gas and leveraging the hydrodynamic cavitation principles at the injection assembly, the present systems and methods can be utilized to create molecular oxygen which has a high oxidation potential but is not well understood today.

The present systems drastically reduce the operating cost and improve the overall effectiveness in many applications. For example, in the case of the system disclosed in U.S. Pat. No. 9,315,402, a large pressure drop is required which demands additional energy input. Furthermore, at only 100% saturation, the power required per unit of gas dissolved is 3-4 times that of the presently disclosed systems and methods. Additionally, the prior art systems require more liquid to deliver a similar amount of gas, and thus, the liquid infrastructure in the presently disclosed system is significantly less. In the case of a Speece type system, only 100% saturation is achieved, requiring more power and infrastructure. Furthermore, the basic fundamentals of Speece require a constant liquid flow independent of the gas flow to create the appropriate velocity gradients, which means that when less gas (less than maximum design point) is required for a given process there is not ability to reduce power consumption.

In the case of wastewater treatment, this could entail utilizing the wastewater or activated sludge, creating an oxygenated solution, and delivering it back to enhance aerobic breakdown of organic constituents. The presently disclosed systems and methods would offer significant cost and energy savings over conventional aeration technologies, as well as other side-stream and pressurized type systems. For both disinfection of water and wastewater, the presently disclosed systems and methods would allow for small systems to be developed requiring minimal power input and no chemical use while providing safe and reliable water in developing countries. At a larger scale, the disclosed systems and methods can be optimized to balance energy and gas use to provide cost-effective disinfection without many of the harmful by-products presented by chlorine and other disinfectants. For agriculture, the systems can be leveraged to provide both the proper amount of oxygen and carbon dioxide to plants, while also providing disinfection capabilities to minimize disease and fungus. The systems can be used to provide clean drinking water to livestock as well. Aquaculture systems require significant amounts of oxygen to support growth of fish and crustaceans, and the disclosed systems would allow for higher growth rates and lower mortality rates in conventional aquaculture systems as well as recirculating systems.

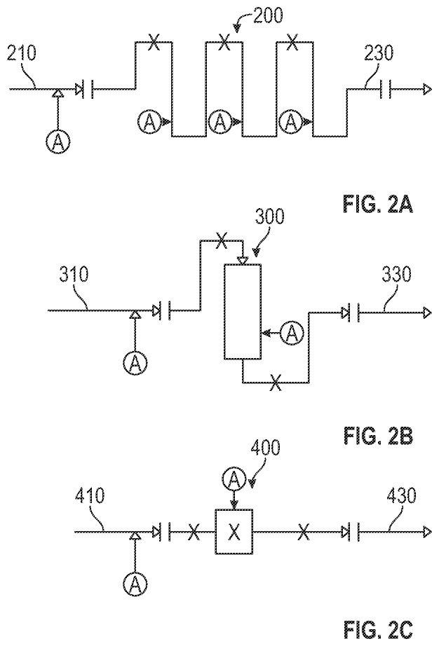

Referring now to FIG. 2A-2C which illustrate several embodiments of reactors which can be used in Step 1 (S1) of the disclosed method. In FIG. 2A a serpentine reactor design 200 is illustrated which can be used during step 1 of the process to mix the liquid and gas. As shown in this figure, liquid is introduced at the input end 210 of the reactor 200 and gas is introduced at the input end and into the liquid flow at various points along the path. The location of the gas input is identified by reference letter “A”. Each time gas is added to the solution, the mixture is subject to cavitation or turbulence in order to aid in the dissolution of the gas into the solution. The location of the cavitation is identified with the reference letter “X”. Ideally the system will operate with target velocities of 20 feet per second (FPS) to 40 FPS where the gas is introduced; target velocities of 10 FPS to 20 FPS at the discharge outlet 230 where the solution exits at the desired concentration; and target velocities of 30 FPS to 100 FPS where induced cavitation is desired.

FIG. 2B illustrates a downflow reactor design 300 which can be used during step 1 of the process. Similar to the reactor 200 shown in FIG. 2A, liquid is introduced at the input end 310 of the system and a gas is inserted at various points in the flow path and cavitation is applied to the flow at several locations in order to aid in dissolving the gas into the liquid. Similar to FIG. 2A, the location of the gas input is identified by reference letter “A”. Each time gas is added to the solution, the mixture is subject to cavitation or turbulence in order to aid in the dissolution of the gas into the solution. The location of the cavitation is identified with the reference letter “X”.

Lastly, FIG. 2C illustrates a inline reactor design 400 which can be used during step 1 of the process. Again, similar to the reactor shown in FIG. 2A, liquid is introduced at the input end 410 of the system and a gas is inserted at various points in the flow path and cavitation is applied to the flow at several locations in order to aid in dissolving the gas into the liquid. Similar to FIG. 2A, the location of the gas input is identified by reference letter “A”. Each time gas is added to the solution, the mixture is subject to cavitation or turbulence in order to aid in the dissolution of the gas into the solution. The location of the cavitation is identified with the reference letter “X”.

Those skilled in the art will readily appreciate that the number, amount and location of the gas input can vary depending upon the intended application for the mixture without departing from the scope of the present invention. Similarly, the number of locations in which cavitation occurs can also vary.

As noted above, in Step 1 of the process, liquid is moved through the reactor/vessel that is pressurized and gas is mixed into the liquid via turbulence or cavitation within the vessel. The process is controlled such that the gas and liquid solution leaving the pressure vessel has a known concentration, generally described as sub saturated, saturated, or supersaturated.

The desired concentration of the solution is selected, calculated and known based on the particular application and process. An exact and precise amount of water is input into a pressurized vessel via gravity or pumping. An exact and precise amount of a particular gas is input into the same pressurized vessel via vacuum or positive pressure. The resulting solution has the exact and precise concentration desired.

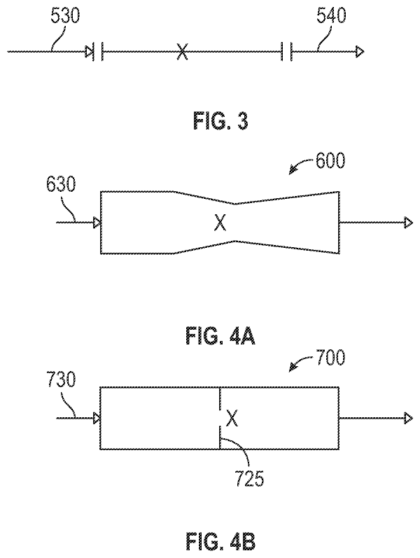

FIG. 3 provides a representation of step 2 of the method of FIG. 1. In step 2, the solution resulting from step 1 which is received at 530 is maintained at the desired concentration until delivered back to the process in step 2 via an injection assembly at output 540. Again, the location of the cavitation is identified with the reference letter “X”

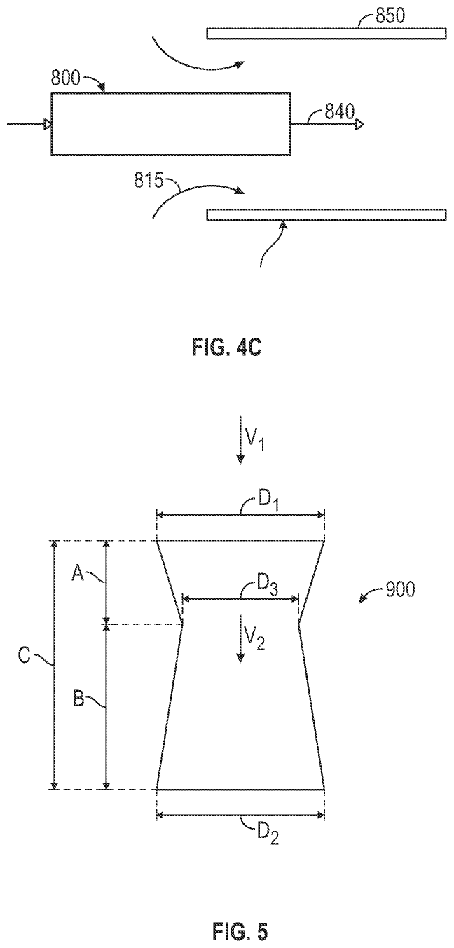

FIG. 4A illustrates an embodiment of a device 600 which can be used to create cavitation in the flow during step 2 of the FIG. 1 process. In this device a gradually reduced diameter increases the velocity and pressure of the fluid until it reaches the section where the diameter increases somewhat abruptly causing a pressure drop and cavitation.

FIG. 4B illustrates a second embodiment of a device 700 which can be used to create cavitation in the flow during step 2 of the FIG. 1 process. As shown in this figures, a orifice plate 725 for example, is inserted into the flowpath in order to sharply reduce the flow area and create cavitation downstream of the plate.

FIG. 4C illustrates an entrainment collar 850 which can be used for mixing the solution 840 with a bulk fluid 815 or process.

Various combinations of the devices shown in FIGS. 4A-4C can be used in step 2 of the process depending upon the application and the desired solution properties. For example, various combinations of the devices shown in FIGS. 4A and 4B can be used in order to optimize cavitation characteristics for hydroxyl radical formation. Alternative arrangements known in the art for creating cavitation or turbulence may also be used in step 2. Various combinations of the devices shown in FIGS. 4A-4C may also be used in step 1 of the process to create cavitation or turbulence, as may alternative arrangements known in the art for creating cavitation or turbulence.

FIG. 5 illustrates a gas/liquid mixing nozzle 900 which can be used to create cavitation or turbulence in the flow through step 1 (S1) of FIG. 1 including, but not limited to, the reactors of FIGS. 2A-2C (one or more cavitation at X) and in the flow through step (S2) of FIG. 1 including, but not limited to, FIG. 3 (cavitation at X). V1 represents the entrance flow velocity and V2 represents the flow velocity exiting the narrow region of the nozzle. D1 represents the diameter at the entrance of nozzle 900, D2 is the diameter at the nozzle exit and D3 is the diameter at the narrowest point in the nozzle. “A” is the distance from the nozzle entrance to the narrowest point. “B” is the distance from the nozzle exit to the narrowest point. And “C” is the length over the nozzle. As examples only, D1:D2=0.5-2.0; D1:D3=3-5; A:B=0.5-1.5; V1:V2=0.1-1.0

It is envisioned that alternative constructions can be created without departing from the scope of the present invention. For example, the system can include various levels of automation and control. In certain constructions, electronic flowmeters, pressure gauges, control valves, along with a programmable logic could be added which would allow for further optimization of the process as well as logging and trending of data.

The system could be modified to address a mixture of gases and liquids. In some applications, it could be desirable to dissolve a particular mixture of gases. Key design parameters and operational controls could be modified for such purposes.

Moreover, the system could be modified to recover energy. For example, by including turbine type generators, liquid flow and residual pressure could be harnessed to generate electricity. In a gravity arrangement, the system might be a producer of energy. In a pumped arrangement, a portion of the energy input to the liquid could be recovered.

Still further, the system could be modified to recover a portion of undissolved gas for other beneficial use. In certain applications, the system will be operating in a contained environment, opportunities exists to capture any undissolved gas. In some applications, this gas can be captured while still under pressure and either be reintroduced to the system or used elsewhere for beneficial use. As an example, a unit operating in an activated sludge system could recover oxygen that could be diverted to an aerobic digester in order to reduce sludge quantities and volumes.

Moreover, the system could be modified to increase hydrodynamic cavitation within the vessel and at the point of reintroduction of the solution to further increase treatment and cost effectiveness for a given application. Still further it is envisioned that the system could be modified to increase the formation of hydroxyl radicals at the point of reintroduction of the solution to further increase treatment and cost effectiveness for a given application.

In certain application, the system could be modified to increase the formation of molecular oxygen at the point of reintroduction of the solution to further increase treatment and cost effectiveness for a given application. For example, by inducing multiple instances of pressure drop and energy transformation, both in the reactor and within the injection assembly, hydrodynamic cavitation can be controlled. In some applications, smaller amounts of cavitation can be leveraged to induce shear forces into the liquid. In the case of biological waste treatment, these forces can be used to lyse cell walls providing for less sludge to be generated from the process. In the case of water treatment, this cavitation can provide disinfection by effectively killing microorganisms.

Furthermore, at higher levels of cavitation, hydroxyl radicals can be produced. These radicals are extremely powerful and effective at oxidation. These radicals can be leveraged to speed up other chemical processes. For example, in the case of odor control, oxygen alone can be an effective treatment, keeping the process aerobic and non-sulfide forming. However, in many cases, the time for oxygen alone to be effective is not realistic. By operating at conditions that promote higher hydroxyl radical formation, the reaction can be catalyzed to lower the required treatment/contact time making a feasible solution to the problem. Additionally, because these radicals have a much higher oxidation potential than oxygen alone, significantly less oxygen can be used, ultimately lowering the total cost of treatment.

Taking this one step further, at extreme levels of cavitation, it is possible to generate molecular oxygen. While oxygen exists in its stable form as O2 in the environment, molecular oxygen (O) is not stable and exhibits even greater oxidation potential than hydroxyl radicals. As an example, the presently disclosed systems could replace ozone disinfection technologies at significantly lower O&M costs. Currently, ozone is generated via electrical charges send through oxygen gas—making about 10% by weight ozone solution. The cost to produce ozone, and inherent safety issues, has limited it adoption for many applications. Leveraging cavitation to product molecular oxygen would allow for even greater oxidation potentials (disinfecting properties) to be generated without the complexity and costs associated with ozone.

As noted above, the presently disclosed systems provide several operational efficiencies over the prior art systems. The data resulting from an analysis of a system which is designed in accordance with the present disclosure and a prior art system is shown below. In the example, oxygen is being used as the gas and the target is to achieve 10,000 lbO2/day. As shown below, the presently disclosed system achieved 116.04 percent saturation and had an operating efficiency of 9.26 lbO2/KW-hr. In contrast, the prior art system had a 26.11 percent saturation and a much lower operating efficiency of 2.08 lbO2/KW-hr.

| | | |
|---|---|---|
| Target Treatment Objective = | 10,000 | lbO2/d |
| Available Energy at Site = | 45 | kW |
| Required Efficiency = | 9.26 | lbO2/kW-hr |
| Assumed Maximum Pressure = | 100 | psi |
| Required Liquid Flow = | 2,776 | gpm |
| Required Energy = | 52.22 | kW |
| Percent Saturation Required = | 116.04 | % Saturation |
| | | SUPERSATURATED |
| Target Treatment Objective = | 10,000 | lbO2/d |
| Available Energy at Site = | 200 | kW |
| Required Efficiency = | 2.08 | 1b02/kW-hr |
| Assumed Maximum Pressure = | 100 | psi |
| Saturated Condition = | 300 | mg/L (Henry's Law, |
| | | steady state) |
| Required Liquid Flow = | 2,776 | gpm |
| Required Energy = | 52.22 | kW |
| Percent Saturation Required = | 26.11 | % Saturation |
| | | SUBSATURATED |

The presently disclosed systems and methods are not targeted at dissolving all of the gas into the liquid, but rather creating a specific solution of gas and liquid in a controlled manner and then delivering that subsaturated, saturated, or supersaturated solution in a controlled manner to accomplish specific objectives.

Moreover, prior art systems which create hydrodynamic cavitation, include a contained reaction vessel where the hydrodynamic cavitation occurs. The presently disclosed systems realize similar benefits without the need for a contained reaction, i.e. in-situ, in a bulk fluid. This allows for significant cost savings at scale and ease of retrofit into existing processes. Furthermore, intense pressures required for prior art systems make them too energy intensive to be used in many cases.

Most of the prior systems that utilize enhanced hydroxyl radical formation include chemical reactions and processes to increase formation. The presently disclosed systems and methods focus on leveraging hydrodynamic cavitation to enhance hydroxyl radical formation without the need for additional chemicals. By combining an oxygen or ozone (gas) and water (liquid) mixture at controlled mass ratios and subjecting that to hydrodynamic cavitation, the presently disclosed systems are able to control the generation of hydroxyl radicals.

What is claimed is:

1. A system for mixing gases and liquids comprising:
i) a reactor vessel including a liquid inlet which receives a predetermined amount of liquid and at least one gas inlet which receives a precise amount of a gas, the reactor vessel including a cavitation device for mixing the gas and liquid to a desired gas concentration; and
ii) an injection assembly.

2. The system as recited in claim 1, wherein the desired gas concentration is subsaturated, saturated or supersaturated.

3. The system as recited in claim 1, wherein the desired gas concentration is selected based on a particular application.

4. The system as recited in claim 1, wherein the reactor vessel is pressurized to at least one atmosphere.

5. The system as recited in claim 1, wherein the reactor is a serpentine reactor and includes more than one location for gas input and following each location of gas input cavitation is created in the mixture.

6. The system as recited in claim 1, wherein the reactor is a downflow reactor and includes more than one location for gas input and following each location of gas input cavitation is created in the mixture.

7. The system as recited in claim 1, wherein the reactor is an inflow reactor and includes more than one location for gas input and following each location of gas input cavitation is created in the mixture.

8. The system as recited in claim 1, wherein the injection assembly has an inlet diameter, an exit diameter and a neck diameter which is less than the inlet diameter.

9. The system as recited in claim 1, wherein the injection assembly includes an orifice plate for creating cavitation in the flow.

10. The system as recited in claim 1, further comprising an entrainment collar.

11. The system as recited in claim 1, wherein the cavitation device includes a nozzle having a nozzle entrance and a nozzle exit with a nozzle neck positioned therebetween, said nozzle entrance and nozzle exit each having a diameter that is larger than a diameter of the nozzle neck.

12. The system as recited in claim 1, wherein the cavitation device includes an orifice.

13. The system as recited in claim 12, wherein the cavitation device includes a plate with the orifice positioned therein.

14. The system as recited in claim 1, wherein the cavitation device includes an entrainment collar.

15. The system as recited in claim 1 wherein the cavitation device is configured to provide hydrodynamic cavitation.

16. The system as recited in claim 15 wherein the injection assembly is configured to provide hydrodynamic cavitation.

17. The system as recited in claim 1 wherein the injection assembly is configured to provide hydrodynamic cavitation.

18. The system of claim 1 wherein the injection assembly is configured to form hydroxyl radicals.

19. The system of claim 18 wherein the injection assembly is configured to provide hydrodynamic cavitation.

20. The system of claim 1 wherein the injection assembly is configured to provide no bubbles.

21. The system of claim 1 wherein the injection assembly is configured to provide nano bubbles.

22. The system of claim 1 wherein the injection assembly is configured to provide fine bubbles.

23. A method for mixing gases and liquids comprising:
a. introducing a predetermined amount of liquid into a reactor vessel;
b. introducing a predetermined amount of a gas into a reactor vessel;
c. mixing the liquid and the gas in the reactor vessel using cavitation or turbulence to form a mixture;
d. injecting the mixture of the liquid and the gas into a process.

24. The method of claim 23 further comprising the step of pressurizing the reactor vessel to at least one atmosphere.

25. The method of claim 23 wherein step b. includes injecting the predetermined amount of gas into the reactor vessel in a plurality of different locations.

26. The method of claim 25 wherein the cavitation or turbulence of step c. occurs in a plurality of locations in the reactor vessel, where each of the plurality of cavitation or turbulence locations follows a corresponding one of the plurality of gas injection locations.

27. The method of claim 23 wherein step c. includes inducing hydrodynamic cavitation.

28. The method of claim 23 wherein step d. includes inducing hydrodynamic cavitation.

29. The method of claim 23 wherein step d. includes forming hydroxyl radicals.

30. The method of claim 23 wherein step c. includes forming a supersaturated solution.
